(19) United States
(12) Patent Application Publication
(10) Pub. No.: US 2002/0128626 A1
(43) Pub. Date: Sep. 12, 2002

(54) Body fluid sealing extensible gaskets for personal care products

(76) Inventors: S. Scott Friderich, Alpharetta, GA (US); Jay Sheldon Shultz, Roswell, GA (US)

Correspondence Address: Pauley Petersen Kinne & Erickson, 2800 W. Higgins Road, Suite 365, Hoffman Estates, IL, 60195, US

(21) Appl. No.: 10/017,474

(22) Filed: Dec. 14, 2001

Related U.S. Application Data
(60) Provisional application No. 60/257,766, filed on Dec. 21, 2000.

(51) Int. Cl. A61F 13/15

(52) U.S. Cl. 604/385.28

(57) ABSTRACT

A containment flap for an absorbent article, such as an incontinence garment, has elasticity in the long axis and extensibility of the flap with a low modulus of elasticity in its transverse direction. The long axis tension of the flap has a force vector normalized to the transverse direction when the flap is placed in curvature over the body of a wearer, thereby providing a force for extending the flap in the transverse direction to maintain contact with the body of the wearer when the garment begins to sag, such as may happen due to gravity when the garment is loaded with absorbed bodily fluids. Material suitable for constructing such flaps is further disclosed.

### BACKGROUND OF THE INVENTION

Pant-like absorbent garments, such as diapers and training pants, typically include a pair of leg openings having an elastic portion around each leg opening, and a waist opening having an elastic portion as well. The elastic portions are intended to fit snugly around a wearer's legs to prevent bodily waste, also sometimes called “exudate” herein, from leaking beyond the garment, yet leakage often persists.

A number of different approaches have been taken to reduce or eliminate leakage from absorbent garments. For example, physical barriers, such as elasticized containment flaps, have been incorporated into such absorbent garments. The amount and configuration of absorbent material in the zone of the absorbent garment in which liquid surges typically occur (sometimes referred to as a target zone) have also been modified.

A further approach to decreasing exudate leakage is to increase tension of the elastic portions around each leg opening and the waist opening. The increased tension is often effective, but just as often results in an undesirable red marking on a wearer's skin due to increased pressure on the wearer's skin.

The use of containment flaps has, in the past, been somewhat limited because the flaps are of a finite width in their transverse direction, meaning that as the absorbent garment becomes loaded with absorbed bodily wastes and sags or droops due to gravity, the gasket will pull away from the wearer's body, sometimes called “a loss of vertical fit”, thereby providing an unwanted leakage path to the exterior of the garment. Providing “oversized” gaskets to try and accommodate a range of sagging is known in the art but results in an undesirable increase of fabric usage from both economical and comfort standpoints.

There is a need or desire for transversely stretchable material suitable for gaskets or containment flaps in absorbent garments that seal fluid within the absorbent garments and which adjust in the transverse direction to the level of sagging in a loaded garment in order to maintain gasketing.

### SUMMARY OF THE INVENTION

The present invention is directed to an improved construction of gaskets in pant-like absorbent garments, and material for those garments, such as, but not limited to, diapers, swim wear, adult incontinence garments, and training pants. The resulting garment may then have extensible gaskets that provide greater leakage protection by maintaining contact with the body of the wearer by extending in their transverse direction when the garment sags away from the body. The present invention provides a material which is at least transversely stretchable in its cross machine direction. The material is suitably light weight and inexpensive. In certain aspects of the invention the material includes thermoplastic fibers of the meltblown or spunbond type, or both. The fibers may be substantially continuous fibers. The nonwoven material made from the thermoplastic fibers may be necked to provide the transverse direction extensibility. A low Young's modulus of up to about 4.5 psi%, and desirably between about 0.90 psi/% to about 4.5 psi/%, may be suitably achieved in one axis of the material by practice of the present invention.

Extensible gaskets may be achieved by using flap material which is extensible, and with a low modulus of elasticity, in a transverse direction, or axis, of the containment flap while being tensioned in the long direction, or axis, of the flap. The flap may be provided with different bands of tension through the selective application of elastics, a so-called “targeted elastic” approach which may use various means to achieve a differential elasticity over the material. Such means may include different types or sizes of elastic elements applied to, or made integral with the flap material. The long axis tensioning of the flap provides a force vector normalized to the transverse direction when the flap is placed in curvature over the body of a wearer, thereby providing a force for extending the flap in the transverse direction. Thus less fabric may be used, contributing to economical manufacture and increased wearer comfort since additional gasketing is only provided on demand.

The transverse direction elastic modulus of the selected flap material should be sufficiently low so that the normal force produced by the long axis elasticity can readily extend the flap material in the transverse direction from, for example, 0% elongation until its point of failure, without a measurable increase in normal force. It will be appreciated that because loss of vertical fit is an irreversible process, the flap material requires very little recovery.

A disposable garment according to one aspect of the present invention may include gasketing provided by elasticized flap portions which are connected to the interior of the garment along the lower part of each leg opening. Throughout use, the elasticized flap portions fit snugly against the wearer and effectively block most spillage of waste material from the leg openings.

It will be appreciated that when flap portions are used for the leak guards, a separate manufacturing step can be required to attach the flap material to the garment. Generally, the flaps have been joined via seams. During active use, some separation at the seams can occur, resulting in failure of the flaps to serve as effective leak guards. Providing a seam which is both leakproof and durable has been challenging, and has added to manufacturing costs. To solve this problem, seamless leak guards were disclosed in co-pending U.S. application Ser. No. 09/290,414, of common ownership herewith. The present invention is also applicable to the integral, or seamless, method of providing gasketing.

As described in the co-pending application, instead of using flaps, seamless leak guards may be provided by extending the liquid-impermeable outer cover layer substantially beyond the absorbent layer on both sides, and to a higher location on the garment and on the wearer. The outer cover extensions on both sides can be reinforced at their edges by elastic leg bands which pull the outer cover extensions upward and away from the absorbent layer, and against the wearer's body. The lateral extensions of the outer cover material, combined with the upward pulling of the elastic leg bands, may provide the garment with seamless leak guards not requiring separately attached flaps.

### BRIEF DESCRIPTION OF THE DRAWINGS

FIG. 1 is a vector diagram of tension and pressure at a point along a curved surface;

FIG. 2 is a front perspective view of a known absorbent garment given as an environment of the present invention;

FIG. 3 is a top plan view of an absorbent garment assembly;

FIG. 4 is a simplified top plan view of the garment indicating tensioning of the flaps in their long axis and extensibility of the flaps in their transverse axis;

FIG. 5 is a cross-sectional view of the absorbent garment assembly, taken along line 5—5 in FIG. 4, and illustrating transverse extensibility of the gasket;

FIG. 6 is a schematic side view of the garment, and a wearer thereof, illustrating the extensible flap material gasketing in response to a loading of the garment with absorbed fluids.

FIG. 7 is a schematic top view of another embodiment wherein the flap is integral with the chassis.

FIG. 8 is a schematic side view of another embodiment wherein the flap has a folded construction.

FIG. 9 is a schematic top view of garment with the flap of FIG. 8.

FIG. 10 is an end view of the folded flap construction taken along line 10-10 of FIG. 9.

FIGS. 11 and 12 illustrate alternative placement of the folds in garment construction.

FIG. 13 is a schematic of the wire weave pattern of thermal bond for the laminate of FIG. 13.

### DEFINITIONS

Within the context of this specification, each term or phrase below will include the following meaning or meanings.

“Article” refers to a garment or other end-use article of manufacture, including but not limited to absorbent articles such as diapers, training pants, swim wear, absorbent underpants, adult incontinence articles, feminine hygiene articles, and medical garments and wraps.

“Attached” can refer to either an integral part or a part joined by a separate joining process.

A “barrier” material is a material with no measurable transmission of a selected substance through that material over the expected term of use of the material.

“Bicomponent” nonwoven filaments are known in the art generally as thermoplastic filaments which employ at least two different polymers combined together in a heterogeneous fashion. Instead of being homogeneously blended, two polymers may, for instance, be combined in a side-by-side configuration, so that a first side of a filament is composed of a first polymer “A” and a second side of the filament is composed of a second polymer “B.” Alternatively, the polymers may be combined in a sheath-core configuration, so that an outer sheath layer of a filament is composed of a first polymer “A,” and the inner core is composed of a second polymer “B.” Other heterogeneous configurations are also possible.

“Bonded” refers to the joining, adhering, connecting, attaching, or the like, of two elements. Two elements will be considered to be bonded together when they are bonded directly to one another or indirectly to one another, such as when each is directly bonded to intermediate elements.

The term “cloth” includes, but is not limited to, a fabric made of fibrous material, commonly a woven fabric of, for example, cotton. Furthermore, the term “cloth” shall also include all nonwoven materials exhibiting a cloth-like feel.

“Connected” refers to the joining, adhering, bonding, attaching, or the like, of two elements. Two elements will be considered to be connected together when they are connected directly to one another or indirectly to one another, such as when each is directly connected to intermediate elements.

“Disposable” refers to articles which are designed to be discarded after a limited use rather than being laundered or otherwise restored for reuse.

“Disposed”, “disposed on”, and variations thereof are intended to mean that one element can be integral with another element, or that one element can be a separate structure bonded to or placed with or placed near another element.

“Elastic”, “elasticized”, “elastomeric” and “elasticity” mean that property of a material or composite by virtue of which it tends to recover its original size and shape after removal of a force causing a deformation.

“Extensible” or “extendible” implies extension under a deformation force with little or no recovery of the original size or shape after the deformation force is removed. A “low modulus of elasticity” with respect to an extensible material implies that little force is required to extend the material and is not meant to imply that the extensible material exhibits elasticity.

“Fabrics” is used to refer to all of the woven, knitted and nonwoven fibrous webs.

“Film” refers to a thermoplastic film made using a film extrusion and/or foaming process, such as a cast film or blown film extrusion process.

“Gaskets”, also called “cuffs” or “containment flaps”, in some instances, are structures within, or on, the personal care product serving as barriers to the escape of bodily exudates. The terms “gaskets”, “flaps” and “containment flaps” will be used interchangeably throughout the application.

“Integral” or “integrally” is used to refer to various portions of a single unitary element rather than separate structures bonded to or placed with or placed near one another.

“Layer” when used in the singular can have the dual meaning of a single element or a plurality of elements.

“Liquid impermeable”, when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable” when used herein.

“Longitudinal” and “transverse” have their customary meaning, as indicated by the longitudinal and transverse directions depicted in FIG. 4 at arrows 62 and 66, respectively. The longitudinal, or long, axis lies in the plane of the article and is generally parallel to a vertical plane that bisects a standing wearer into left and right body halves, when the article is worn. The transverse axis lies in the plane of the article generally perpendicular to the longitudinal axis. The article and its parts, although illustrated as longer in the longitudinal direction than in the transverse direction, need not be so.

“Machine direction”, or MD, refers to the length of a fabric in the direction in which it is produced, as opposed to “cross direction”, or CD, which refers to the width of a fabric in a direction generally perpendicular to the machine direction.

“Meltblown fiber” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are desirably substantially continuous in length.

“Member” when used in the singular can have the dual meaning of a single element or a plurality of elements.

As used herein, the term “necked material” refers to any material which has been drawn in at least one dimension, (e.g. lengthwise), reducing the transverse dimension, (e.g. width), such that when the drawing force is removed, the material can be pulled back, or relax, to, or near, its original width. The necked material typically has a higher basis weight per unit area than the un-necked material. When the necked material returns to its original un-necked width, it should have about the same basis weight as the un-necked material. This differs from stretching/orienting a material layer, such as a film, during which the layer is thinned and the basis weight is permanently reduced.

The term “nonwoven fabric” or “nonwoven web” means a web having a structure of individual fibers or threads which are interlaid, but not in a regular or identifiable manner as in a knitted fabric. Nonwoven fabrics or webs have been formed from many processes such as, for example, meltblowing processes, spunbonding processes, air-laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

“Permanently bonded” refers to the joining, adhering, connecting, attaching, or the like, of two elements of an absorbent garment such that the elements tend to be and remain bonded during normal use conditions of the absorbent garment.

The term “personal care absorbent product” includes without limitation diapers, training pants, swim wear, absorbent underpants, baby wipes, adult incontinence products, and feminine hygiene products.

Words of degree, such as “About”, “Substantially”, and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al. Spunbond fibers are quenched and generally not tacky when they are deposited onto a collecting surface. Spunbond fibers are generally continuous and often have average deniers larger than about 0.3, more particularly, between about 0.6 and about 10.

As used herein, the term “substantially continuous fibers” refers to fibers, including without limitation, spunbond and meltblown fibers, which are not cut from their original length prior to being formed into a nonwoven web or fabric. Substantially continuous fibers may have average lengths ranging from greater than about 15 centimeters to more than one meter, and up to the length of the web or fabric being formed. The definition of “substantially continuous fibers” includes fibers which are not cut prior to being formed into a nonwoven web or fabric, but which are later cut when the nonwoven web or fabric is cut, and fibers which are substantially linear or crimped.

“Thermoplastic” describes a material that softens when exposed to heat and which substantially returns to a nonsoftened condition when cooled to room temperature.

A “transversely” stretchable, or extendible, material is one which extends more easily along a first axis than along a second axis. “Transversely” extendible is not necessarily meant to imply that there is no extendibility in the second axis.

These terms may be defined with additional language in the remaining portions of the specification.

### DETAILED DESCRIPTION OF THE INVENTION

It is well known that pressure exerted from elastic tension at a given body contact point is proportional to the curvature at the point as well as to the amount of tension, as demonstrated by the LaPlace equation:

Pg=σ₁/R₁ (1)

where Pg is the normal force or gasket pressure, R₁ is the radius of curvature along a wearer's body 5, and σ₁ is the tension in the tangential direction (see FIG. 1). Thus, it can be appreciated that pressure Pg is generated under a given tension σ₁. Pg is a force aligned in the transverse direction of the containment flap providing a force to extend the flap as further explained below.

In pant-like absorbent garments having elasticized leg openings and/or an elasticized waist opening, the elastic tension σ₁ should be high enough so that sufficient pressure Pg is exerted at all points around the perimeter of the opening, to seal the garment against the wearer's body. This force in the present invention should also be in an amount to easily achieve fall extension of the flap material towards the wearer.

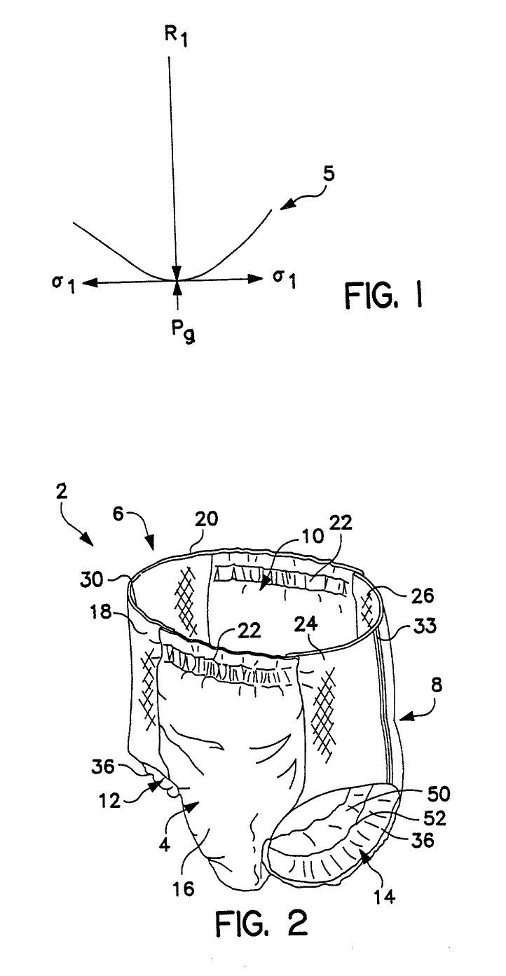

Referring to FIG. 2, a conventional pant-like absorbent garment 2 for use in conjunction with the present invention includes a waste containment section 4 and two side portions 6 and 8 defining a waist opening 10 and a pair of leg openings 12 and 14. The side portion 6 includes stretchable panels 18 and 20 joined together at seam 30. The side portion 8 includes stretchable panels 24 and 26 joined together at seam 33. Seams 30 and 33 extend longitudinally from the waist opening 10 to the leg openings 12 and 14 of the garment 2.

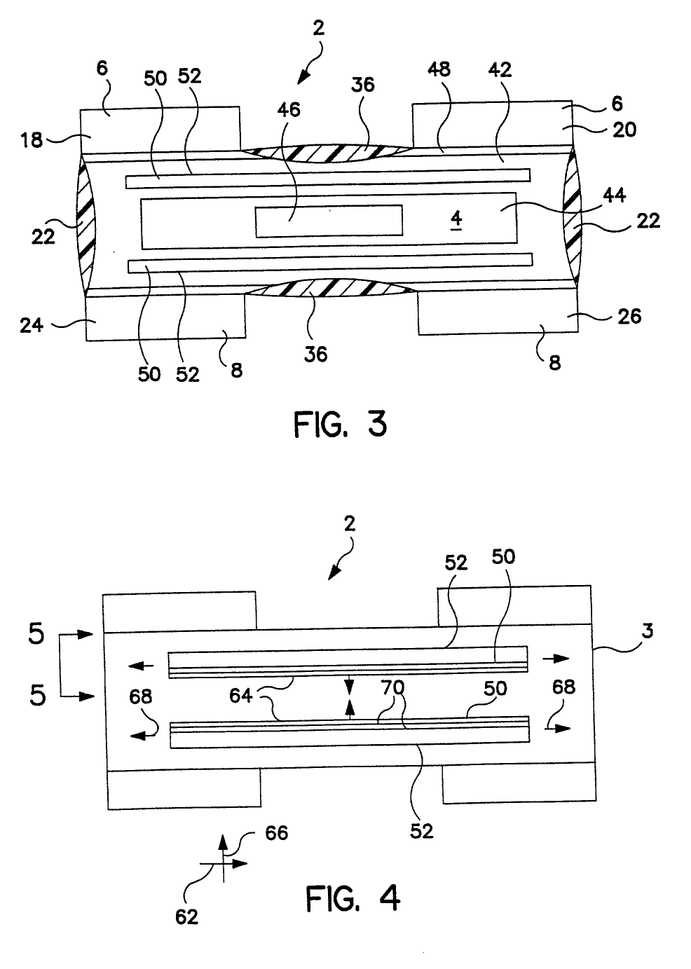

The waste containment section 4 includes multiple layers, as shown in FIG. 3, including, for instance, a liquid-permeable body side liner 42, an absorbent core layer 44, a surge layer 46, and a liquid-impermeable outer cover 48 which faces away from the wearer. The waste containment section 4 includes waist elastics 22 on the front and back of the garment 2. The leg openings 12 and 14 also include leg elastics 36 which extend substantially around the portion of the leg openings defined by the waste containment section 4.

The stretchable side portions 6 and 8 can be constructed of conventional woven or nonwoven materials, formed from a wide variety of elastic and stretchable polymers. Suitable polymers include without limitation block copolymers of polystyrene, polyisoprene and polybutadiene; copolymers of ethylene, natural rubbers and urethanes; and combinations of the foregoing. Particularly suitable are styrene-butadiene block copolymers which have been sold by Shell Chemical Co. under the trade name KRATON®. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. Also suitable are coextruded composites of the foregoing, and elastomeric staple integrated composites where staple fibers of polypropylene, polyester, cotton and other materials are integrated into an elastomeric meltblown web. Certain elastomeric ultra-low density olefin polymers such as single-site or metallocene-catalyzed olefin polymers and copolymers are also suitable for the side portions 6 and 8. Referencing FIGS. 2 and 3, the stretchable side portions 6 and 8 are desirably rectangular in shape, and desirably, as shown in FIG. 2, extend from the top of the waist opening 10 to the leg openings 12 and 14. The side portions 6 and 8 may also be laminates of multiple layers, and are desirably breathable to water vapor but impervious to liquids.

When a laid open absorbent garment as shown in FIG. 3 is assembled into the absorbent garment shown in FIG. 2, the longitudinal seams 30 and 33 may be formed by conventional methods including, without limitation, ultrasonic welding, thermal bonding, adhesive bonding, stitch bonding and the like. Ultrasonic welding is a presently desirable technique. The various bonding techniques are conventional, and are neither critical nor limiting as to the present invention.

The leg elastics 36 may be attached to the outer cover 48 by a variety of techniques including adhesive bonding, ultrasonic bonding, thermal bonding, stitch bonding or other conventional techniques. Suitable adhesives include spray adhesives, hot melt adhesives, self-adhering elastomeric materials and the like. Often, the leg elastics 36 will be applied in the stretched condition to the outer cover 48, and then allowed to retract, causing gathering of the outer cover 48 when the leg elastics 36 are retracted. The leg elastics 36 desirably comprise at least two elastic bands, more desirably at least four elastic bands.

In the vicinity of the waist opening 10, the waist elastics 22 may be attached to or embedded within the garment 2. The waist elastics 22 may include single or multiple elastic bands constructed from any of the same materials as the leg elastics 36. The waist elastics 22 in the front and back of the garment 2 desirably have lengths which are nearly the same, or slightly shorter than the width of the outer cover 48. The waist elastics 22 may be attached to the outer cover 48 using the same techniques as for attaching leg elastics 36.

A wide variety of elastic materials may be employed for the leg elastics 36 and the waist elastics 22. Examples include a film or meltblown web formed using block or graft copolymers of butadiene, isoprene, styrene, ethylene-methyl acrylate, ethylene-vinyl acetate, ethylene-ethyl acrylate or blends thereof. One desirable elastomer is a block copolymer of styrene-ethylbutadiene-styrene. Polyester elastomeric materials, polyurethane elastomeric materials and polyamide elastomeric materials can be used as well. Elastomeric ultra-low density olefin polymers such as single-site or metallocene-catalyzed olefin polymers and copolymers can also be employed. Also, the leg elastics 36 and the waist elastics 22 can be made of an activatable material applied in an unstretched condition, and activated by heat, light or moisture or radiation to cause shrinkage and elasticity.

As previously indicated, the outer cover 48 may include a single layer, or may include multiple layers joined together. The outer cover 48, as shown in FIG. 3, may include two layers, a cloth layer and a polymer layer, joined by an outer cover adhesive layer. The cloth layer of the outer cover 48 can be made from a wide variety of woven or nonwoven material, films, or a film-coated nonwoven material, including, for instance, cast or blown films of polyethylene, polypropylene, polyester or blends thereof. The cloth layer may also be a composite of a bonded carded or spunbond or meltblown material, for example, a spunbond-meltblown composite of thermoplastic material or a spunbond-meltblown-spunbond thermoplastic material, wherein the spunbond layer can provide a cloth-like texture and the meltblown layer can provide liquid impermeability. Materials of which the cloth layer can be made include nonwovens having a basis weight of about 0.4 ounces per square yard (13.6 gsm) or greater. The polymer layer of the outer cover 48 can include extruded films of polyolefin polymers or copolymers, or other thermoplastic materials.

The outer cover 48, absorbent core layer 44, surge layer 46 and body side liner 42 may also be joined together using ultrasonic bonding, thermal bonding, stitch bonding, or any of the adhesive materials described above for the attachment of the leg elastics 36 and the waist elastics 22.

The absorbent core layer 44 can, without limitation, be made of wood pulp fluff or a mixture of wood pulp fluff and a superabsorbent material, or a wood pulp fluff integrated with a thermoplastic absorbent material treated with a surfactant, or absorbent foams. Thermal binders, such as Pulpex® can be used in blends or layering with the fluff and superabsorbent material. The absorbent core layer 44 can also include a batt of meltblown synthetic fibers, a bonded carded web of synthetic or natural fibers or blends thereof, a composite of meltblown fibers and the like. The synthetic fibers can be, but are not limited to, polypropylene, polyethylene, polyester and copolymers of these or other polyolefins.

Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthum gum, locust bean gum and the like. Mixtures of natural and wholly or partially synthetic superabsorbent polymers can also be useful in the present invention. Other suitable absorbent gelling materials are disclosed by Assarsson et al. in U.S. Pat. No. 3,901,236 issued Aug. 26, 1975. Processes for preparing synthetic absorbent gelling polymers are disclosed in U.S. Pat. No. 4,076,663 issued Feb. 28, 1978 to Masuda et al. and U.S. Pat. No. 4,286,082 issued Aug. 25, 1981 to Tsubakimoto et al.

Both the surge layer 46 and the body side liner 42 are constructed from liquid pervious materials. These layers function to transfer liquid to the absorbent core layer 44. Suitable materials include porous woven materials, porous nonwoven materials, open-celled foams, and apertured films. Examples include, without limitation, any flexible porous sheets of polyolefin fibers, such as polypropylene, polyethylene or polyester fibers; webs of spunbond polypropylene, polyethylene or polyester fibers; webs of rayon fibers; bonded carded webs of synthetic or natural fibers or combinations thereof. Either layer may also be an apertured plastic film. The various layers of the garment 2 have dimensions which vary depending on the size and shape of the wearer.

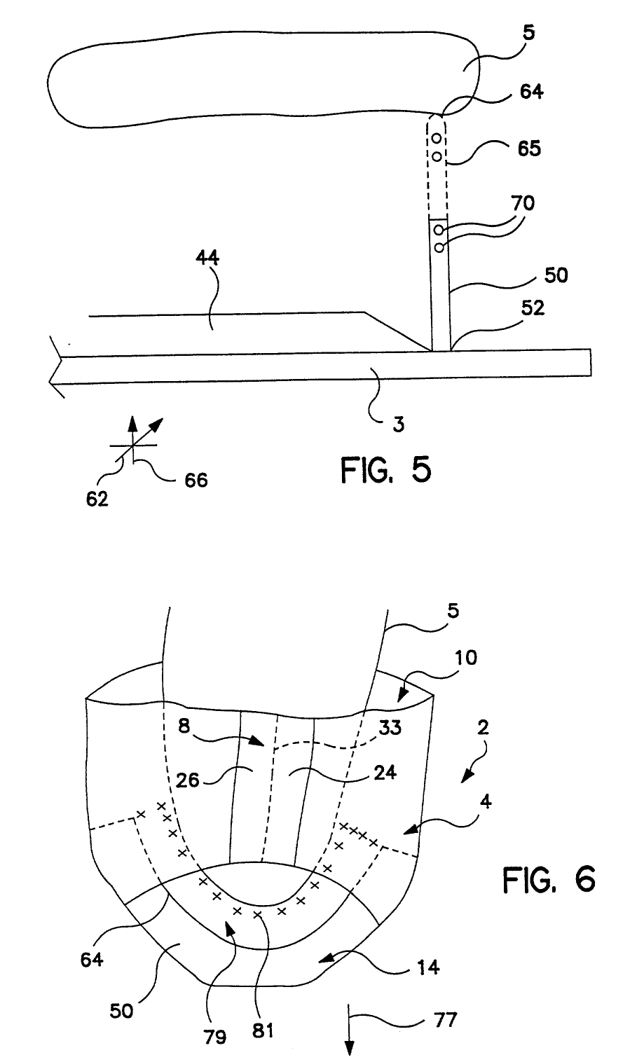

As seen in FIGS. 4-6, the garment 2 according to the present invention will have the flaps 50, hereinafter described in the singular, extending in a long axis, or direction, corresponding to the longitudinal axis or direction 62 of the garment 2. The flap 50 will have an attached edge 52 attached, i.e. affixed to, or integral with, the garment, and a free edge 64 for contacting the body of the wearer 5 (FIGS. 5 and 6). The flap 50 will have a transverse direction 66 perpendicular to its long direction 62. Arrows 68 indicate the long axis tensioning force σ₁ achieved through addition of elastics 70 extending in the long direction of the flap 50.

As seen in FIG. 5, the gasket 50, in its functional position, extends largely perpendicularly to the garment chassis 3, with chassis 3 defined for present purposes as including all parts of the absorbent article exclusive of the flaps 50. Extension of the gasket is indicated by dotted, or phantom, portion 65. Elastic members 70, or elasticity, may be provided such as discussed above or in any known manner sufficient to provide a normalizing force adequate to extend the flap in the transverse direction 66.

Materials suitable for use in constructing a gasket according to the present invention will satisfy the criteria of being easily extensible and with a low recovery, or modulus of elasticity, in a first direction and readily accepting of elastics, e.g. Lycra (TM) strands or otherwise providing a tensioning force, in a second direction perpendicular to the first as discussed above. The material should further provide suitable liquid barrier properties to function as a gasket. Additionally a soft feel and other esthetic properties are desirable. Among the materials contemplated for use are spunbond/meltblown/spunbond (SMS) multilayer laminates, spunbond nonwoven webs, and film/nonwoven web laminates, including neck stretched microporous film/nonwoven web laminates such as found in co-pending application Ser. No. 60/201,830 and further discussed below. Indeed many of the list materials, or parts thereof, may be suitably neck stretched in the machine direction during their manufacture to thereby provide an extensibility in their cross machine directions, which will ultimately become the transverse direction of the flap. Films meeting the above criteria may also be used alone or in conjunction with nonwoven webs of crimped fibers. Highly oriented nonwoven fabric such as certain types of bonded carded web may also be used in the context of the present invention.

Referencing especially FIG. 6, it is seen that an absorbent garment 2, upon becoming loaded with absorbent fluid, will sag under the force of gravity 77 away from the body of the wearer 5. This loss of vertical fit ordinarily creates a gap 79 between the gasket 50 and the wearer 5 leading to leakage. However, through provision of the flaps 50 of the present invention, as the garment 2 sags, the flap 50 extends toward the body 5 as indicated at line 81 in order to maintain contact with the body 5 thereby providing gasketing and preventing leakage to the exterior of the garment.

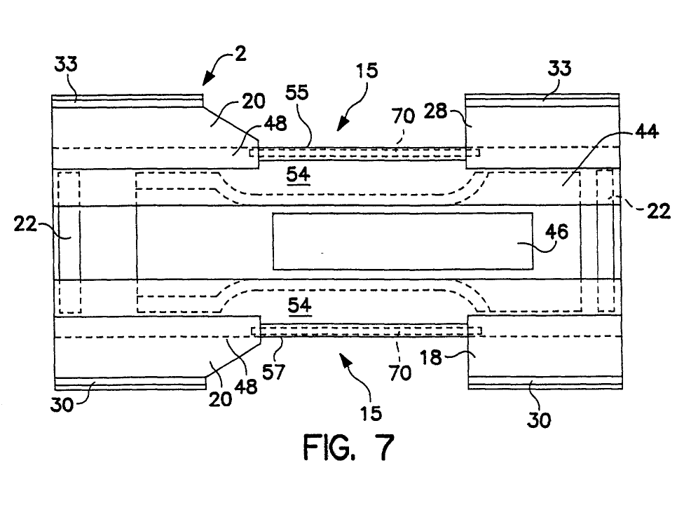

Referencing FIG. 7, in an embodiment wherein portions 54 of the outer cover 48 extend beyond the absorbent layer 44, the extended portions 54 may serve as seamless leak guards. By “seamless”, it is meant that the leak guards are not separately attached and, thus, do not require a seam for attachment to the waste containment section 4. To effectively serve as leak guards, the difference in width between the absorbent layer and the outer cover must be substantial in the central region 15 between the leg openings. Generally, the outer cover 48 is at least about 40% wider than the absorbent layer 44 in the central region 15. Desirably, the outer cover 48 is at least about 60% wider than the absorbent layer 44 in the central region 15. The outer cover 48 in this embodiment would be constructed and arranged from materials including elastics 70, selected according to the above discussed criteria, at least in so far as the gasketing area is concerned.

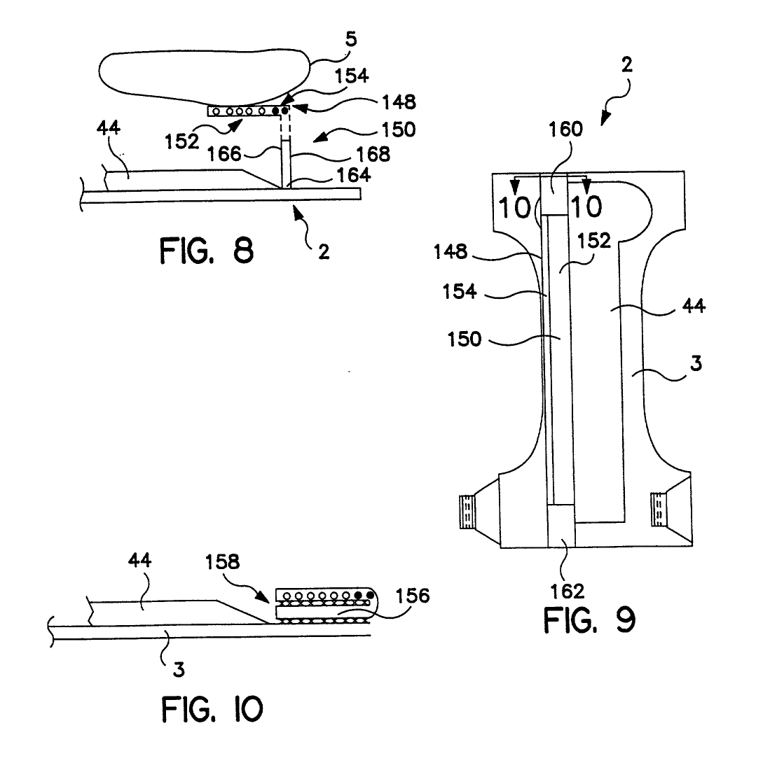

Referencing FIGS. 8-12, another embodiment of the present invention is shown. This embodiment comprises the construction of non-woven and elastic materials to form a flap-like gasket 150 running in the longitudinal direction of an absorbent article 2 and having a fold 148 therein. This flap construction comprises a transversely extensible facing material, a number of lower tension elastic strands 152 to hold the flap 150 next to the skin, and a number of higher tension elastic strands 154 to lift the flap construction to maintain intimate contact with the body surface 5 of the user.

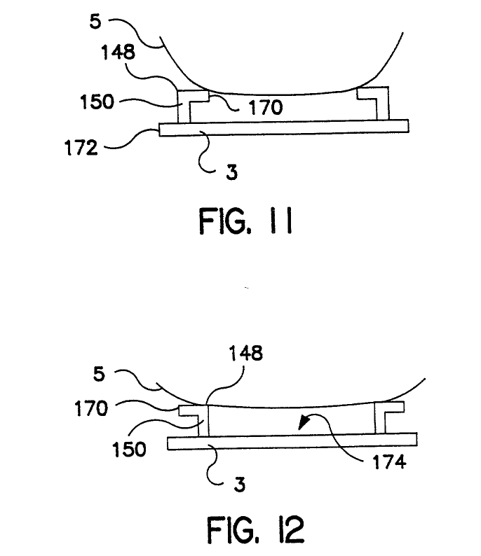

Referencing FIGS. 9 and 10, the gasket 150 is constructed so that the outside face 156 of the gasket portion between the higher tension elastic strands 154 and the garment is glued at its ends 160, 162 to the garment chassis 3. Likewise, the inside faces 158 of the gasket 150 are attached to each other at the ends of the gasket.

Between the longitudinal ends of the garment chassis, the gasket 150 is only attached to the chassis at its attached edge 164, leaving both the inner and outer faces 166, 168 respectively, of the gasket free to move. The material extensibility in the transverse direction between the garment 2 and the higher tension elastic strands 148 allows the region at the loose edge of the flap containing the lower tension strands 152 to continuously interface with the wearer's body 5 throughout the product life.

The extensible gasket material requires a very low elastic modulus in the transverse direction. The higher tension elastics 154 must have sufficient tangential force against the user's body 5 surface to create a normal force in the transverse direction of the flap construction that can readily extend the gasket material from 0% elongation until its point of failure. The extensibility in this portion of the gasket construction keeps the free portion of the gasket between the higher tension elastic and the loose edge firmly against the body surface 5. The lower tension strands 152 further provide extensibility and conformance along the user's body surface.

Referencing FIGS. 11 and 12, it can be seen that the gasket 150 may be attached to the garment chassis 3 so that the fold 148 of the gasket is either on the inside of the garment (FIG. 12) or out from the garment (FIG. 11). By placing the fold 148 out from the garment, i.e. the loose flap edge 170 adjacent the chassis edge 172 (in the folded position), as in FIG. 11, pressure against the gasket forms a shearing force at the interface of the flap and the body surface 5 which is difficult to overcome due to friction. Conversely, placing the fold 148 in toward the garment the loose flap edge 170 adjacent the chassis center-line 174, as in FIG. 12, would create a peel force at the same interface in the same circumstances. Therefore, it is desirable to place the gasket fold outward to help guard against gasket failure and leakage.

With current flap construction, a secure gasket depends on the interface of the flap tip and the body surface for containment. This particular embodiment improves on the known design by increasing the area of interface between the gasket and the body surface and by providing a transverse directional extensibility to maintain a secure gasket between the product and the user's body surface.

Thermal bonding of two material layers together can be accomplished by at least two methods. The first is using heat and pressure as with heated, patterned bonding rolls. Both rolls may be patterned or one may be patterned and the other may be smooth. One or both of the rolls may be heated or a secondary heat source may be used. If conditions dictate, one of the rolls may be cooled. In any event, the heat should be adjusted so that the bonding agent in the film becomes tacky to bond the two layers together while still maintaining the temperature at least about 5° C. cooler than the melting point of the primary predominately linear polyolefin polymer in the film. By “primary” it is meant that predominately linear polyolefin polymer having the highest weight percent of the total weight of the film if there is more than one predominately linear polyolefin polymer in the film polymer blend.

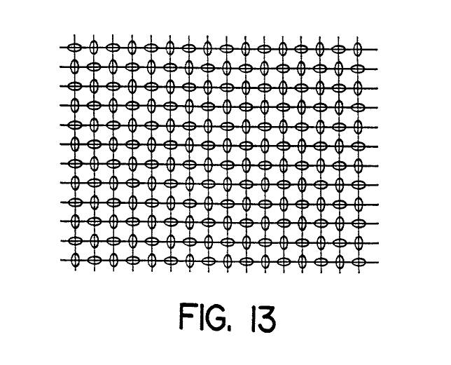

Bond patterns and area may be varied depending upon the particular end-use. Suitable bond patterns can include point bonds, continuous lines, decorative patterns and combinations of the foregoing and may include the wire weave bond pattern illustrated in FIG. 13. Bond area will depend upon the degree of lamination desired. For personal care absorbent article applications, bonding should be sufficient to require at least a 5 gram load to delaminate the two layers.

The second method of bonding is ultrasonic bonding which also is well known to those having ordinary skill in the art. The anvil roll of an ultrasonic bonder can be designed with any of several bond patterns.

Transversely stretchable, or extendible, materials suitable for use with the present invention are presented below. The exemplary materials are presented as contemplated means of accomplishing certain aspects of the invention and are not intended to limit the scope of the invention. Cross direction, or CD, Young's modulus and machine direction, or MD, Young's modulus, were measured to indicate extendibility in those axes. The CD and MD Young's moduli are then reported as a ratio to indicate strength and flexibility of the sample materials. Hydrohead is also reported as an indication of the absolute liquid barrier properties of the materials.

Material Examples

### EXAMPLE 1

A necked spunbond comprising of a 0.4 osy layer of PRISM bicomponent spunbond fibers, as taught in U.S. Pat. No. 5,382,400 to Pike et al., and necked to about 45% of its original width, was tested according to the below listed test procedures and found to have a CD Young's modulus of 2.97 psi/%, an MD Young's modulus of 87.73 psi/%, an MD/CD Young's modulus ratio of 29.54, and a hydrohead of 3.67 mbar.

### EXAMPLE 2

A spunbond/meltblown/spunbond laminate comprising two spunbond layers of a 0.4 osy layer of PRISM bicomponent spunbond fibers, as taught in U.S. Pat. No. 5,382,400 to Pike et al., and necked to about 45% of their original width, with a 0.2 osy layer of meltblown Kraton G filaments between the spunbond layers, was tested according to the below listed test procedures and found to have a CD Young's modulus of 3.70 psi/%, an MD Young's modulus of 91.02 psi/%, an MD/CD Young's modulus ratio of 24.60, and a hydrohead of 11.33 mbar.

### EXAMPLE 3

A spunbond/meltblown laminate comprising a single spunbond layer of a 0.4 osy un-necked layer of PRISM bicomponent spunbond fibers, as taught in U.S. Pat. No. 5,382,400 to Pike et al., with a 0.2 osy layer of meltblown KRATON G filaments laminated to the spunbond layer, was tested according to the below listed test procedures and found to have a CD Young's modulus of 28.89 psi/%, an MD Young's modulus of 0.90 psi/%, an CD/MD Young's modulus ratio of 32.10, and a hydrohead of 14.50 mbar. It will be appreciated that the low Young's modulus direction of this material example is ninety degrees different from the previous examples, but the material may still be suitable for use with certain aspects of the present invention.

Test Methods

Elongation Testing:

A one inch strip of each material was evaluated on an Instron automated stress-strain tester. Specifically, the gap size between clamps on each side of the material during the stress-strain test was set at 0.25 inches. A cross-head, or clamp separation, speed of 20 in/min was used. A maximum elongation of 200%, i.e. specifically from ¼ inch to ¾ inch where samples did not break. A maximum load: of 30 pounds was permitted. This procedure was used to measure the CD Young's modulus as well as the MD Young's modulus of the materials. Hydrohead testing:

In this test, water pressure is measured to determine how much water pressure is required to induce leakage in three separate areas of a test material. The water pressure is reported in millibars (mbars) at the first sign of leakage in three separate areas of the test specimen. The pressure in millibars can be converted to hydrostatic head height in inches of water by multiplying millibars by 0.402. Pressure measured in terms of inches refers to pressure exerted by a number of inches of water. Hydrostatic pressure is pressure exerted by water at rest.

Apparatus used to carry out the procedure includes a hydrostatic head tester, such as TEXTEST FX-3000 available from ATI Advanced Testing Instruments Corp. of Spartenburg, S.C., a 25.7 cm² test head such as part number FX3000-26 also available from ATI Advanced Testing Instruments Corp., purified water such as distilled, deionized, or purified by reverse osmosis, a stopwatch accurate to 0.1 second, a one-inch circular level, and a cutting device, such as scissors, a paper cutter, or a die-cutter.

Prior to carrying out this procedure, any calibration routines recommended by manufacturers of the apparatus being used should be performed. Using the cutting device, the specimen is cut to the appropriate size. Each specimen has a minimum size that is sufficient to allow material to extend beyond the outer diameter of the test head. For example, the 25.7 cm² test head requires a 6-inch by 6-inch, or 6-inch diameter specimen. Specimens should be free of unusual holes, tears, folds, wrinkles, or other distortions.

First, make sure the hydrostatic head tester is level. Close the drain faucet at the front of the instrument and pull the upper test head clamp to the left side of the instrument. Pour approximately 0.5 liter of purified water into the test head until the head is filled to the rim. Push the upper test head clamp back onto the dovetail and make sure the plug is inserted into the socket at the left side of the instrument. Turn the instrument on and allow the sensor to stabilize for 15 minutes. Make sure the Pressure Gradient thumbwheel switch is set to 60 mbar/min. Make sure the drain faucet is closed. The water temperature should be maintained at about 75° Fahrenheit±10° Fahrenheit. Use the Light Intensity adjustment to set the test head illumination for best visibility of water droplets passing through the specimen.

Once the set-up is complete, slide the specimen onto the surface of the water in the test head, from the front side of the tester. Make sure there are no air bubbles under the specimen and that the specimen extends beyond the outer diameter of the test head on all sides. If the upper test head clamp was removed for loading the specimen, push the clamp back onto the dovetail. Pull down the lever to clamp the specimen to the test head and push the lever until it comes to a stop. Press the Reset button to reset the pressure sensor to ZERO. Press the Start/Pause button to start the test. Observe the specimen surface and watch for water passing through the specimen. When water droplets form in three separate areas of the specimen, the test is complete. Any drops that form within approximately 0.13 inch (3.25 mm) of the edge of the clamp should be ignored. If numerous drops or a leak forms at the edge of the clamp, repeat the test with another specimen. Once the test is complete, read the water pressure from the display and record. Press the Reset button to release the pressure from the specimen for removal. Repeat procedure for desired number of specimen repeats.

While the embodiments of the invention described herein are presently considered desirable, various modifications and improvements can be made without departing from the spirit and scope of the invention. The scope of the invention is indicated by the appended claims, and all changes that fall within the meaning and range of equivalents are intended to be embraced therein.

We claim:

1. A material suitable for a transversely extendible containment flap in an absorbent article comprising:
a) the material having a first axis and a second axis,
b) the material having a Young's modulus up to about 4.5 psi/% in the first axis.

2. The material suitable for a transversely extendible containment flap in an absorbent article of claim 1, further comprising: the material having a Young's modulus of between about 29 psi/% to about 95 psi/% in the second axis.

3. The material suitable for a transversely extendible containment flap in an absorbent article of claim 1, further comprising:
a) the material having a CD and an MD,
b) the material having a Young's modulus of between about 3.0 psi/% to about 3.7 psi/% in the CD.

4. The material suitable for a transversely extendible containment flap in an absorbent article of claim 3, further comprising: the material having a Young's modulus of between about 85 psi/% to about 95 psi/% in the MD .

5. The material suitable for a transversely extendible containment flap in an absorbent article of claim 1, further comprising:
a) the material having a CD and an MD,
b) the material having a Young's modulus of about 0.9 psi/% in the MD.

6. The material suitable for a transversely extendible containment flap in an absorbent article of claim 5, further comprising: the material having a Young's modulus of greater than about 29 psi/% in the CD

7. The material suitable for a transversely extendible containment flap in an absorbent article of claim 3, further comprising: the material having an MD:CD Young's modulus ratio of between about 24 and about 30.

8. The material suitable for a transversely extendible containment flap in an absorbent article of claim 5, further comprising: the material having an CD:MD Young's modulus ratio of about 32.

9. The material suitable for a transversely extendible containment flap in an absorbent article of claim 1, further comprising: the material having a basis weight of between about 0.4 osy and about 1.0 osy.

10. The material suitable for a transversely extendible containment flap in an absorbent article of claim 1, further comprising: the material having a basis weight of about 0.6 osy.

11. The material suitable for a transversely extendible containment flap in an absorbent article of claim 3, further comprising: a necked spunbond nonwoven web of substantially continuous bicomponent fibers being necked to about 45% of its original width, the material having a basis weight of about 0.4 osy, a Young's modulus in the CD of about 3 psi/%, and a hydrohead of up to about 4 mbar.

12. The material suitable for a transversely extendible containment flap in an absorbent article of claim 3, further comprising: a spunbond/meltblown/spunbond laminate having two layers of substantially continuous bicomponent spunbond fibers, each being necked to about 45% of their original width and having a basis weight of about 0.4 osy; and an about 8 gsm layer of elastomeric fibers having a basis weight of about 0.2 osy; the material having a Young's modulus in the CD of about 3.7 psi/%, and a hydrohead of greater than about 11 mbar.

13. The material suitable for a transversely extendible containment flap in an absorbent article of claim 5, further comprising: a spunbond/meltblown laminate having a single layer of substantially continuous bicomponent spunbond fibers, the single layer having a basis weight of about 0.4 osy; and an about 8 gsm layer of elastomeric fibers having a basis weight of about 0.2 osy; a Young's modulus in the MD of about 0.9 psi/%, and a hydrohead of up to about 15 mbar.

14. The material suitable for a transversely extendible containment flap in an absorbent article of claim 1, further comprising: the material having a hydrohead of between about 4 mbar and about 15 mbar.

15. An absorbent article comprising:
a) an absorbent chassis, the chassis having a longitudinal axis;
b) a containment flap having a free edge and an attached edge, the attached edge being attached to the chassis, the containment flap having a long axis and a transverse axis, the long axis being parallel to the longitudinal axis of the chassis, the flap having a tensioning force in its long axis, the flap having a low modulus of elasticity in its transverse axis and being extensible in its transverse axis, the tensioning force being sufficient to produce extension of the flap in the transverse direction.

16. The absorbent article of claim 15 wherein: the flap has a modulus of elasticity in its transverse axis below about 5 psi/%.

17. The absorbent article of claim 15 wherein: the flap comprises substantially continuous fibers.

18. The absorbent article of claim 15 wherein: the flap is integral with an extensible outer cover of the article.

19. The absorbent article of claim 15 wherein: the flap is bonded to the chassis of the article.

20. The absorbent article of claim 15 wherein: the flap includes elastomerics within the flap to supply the tensioning force.

21. The absorbent article of claim 15 wherein: the flap includes targeted elastics with higher tension elasticity located on that part of the flap located closer to the chassis and lower tension elasticity located on that part of the flap located farther from the chassis.

22. The absorbent article of claim 15 wherein the flap comprises a material selected from the group comprising: necked spunbond/meltblown/spunbond material and necked spunbond material.

23. The absorbent article of claim 15 wherein: the flap comprises a necked spunbond/meltblown/spunbond material having two layers of substantially continuous bicomponent spunbond fibers, each being necked to about 45% of their original width and having a basis weight of about 0.4 osy; and an about 8 gsm layer of elastomeric fibers having a basis weight of about 0.2 osy; the material having a Young's modulus in the CD of about 3.7 psi/%, and a hydrohead of greater than about 11 mbar.

24. The absorbent article of claim 15 wherein: the flap comprises a necked spunbond material having a necked spunbond nonwoven material of substantially continuous bicomponent fibers being necked to about 45% of its original width, with a basis weight of about 0.4 osy, a Young's modulus in the CD of about 3 psi/%, and a hydrohead of less than about 4 mbar.

25. The absorbent article of claim 15 wherein the flap comprises a spunbond/meltblown laminate having a single layer of substantially continuous bicomponent spunbond fibers, the single layer having a basis weight of about 0.4 osy; and an about 8 gsm layer of elastomeric fibers having a basis weight of about 0.2 osy; the material having a Young's modulus in the MD of about 0.9 psi/%, and a hydrohead of less than about 15 mbar.

26. The absorbent article according to claim 15 wherein:
a) the flap has a first section proximal to the attached edge, the first section being extensible in the transverse axis;
b) the flap has a second section adjacent the first section and having an elasticity in the long axis for supplying the tensioning force; and
c) the flap has a third section proximal to the free edge and adjacent the second section, the third section having a low modulus of elasticity in the long axis for aid in retaining the flap against the body of a wearer.

27. A method of making an absorbent article comprising:
making a material having extensibility with a low first modulus of elasticity in a first direction and elasticity in a second direction;
creating a flap from the material to have a longitudinal axis and a transverse axis with the material first direction oriented in the transverse axis of the flap; and
providing an absorbent chassis with a liner side and an exterior side; and attaching a longitudinal edge of the flap to the absorbent chassis so as to enable the flap to extend outwardly from the liner side and act as a leakage barrier for the absorbent article when worn by a wearer.
